(19) United States
(12) Patent Application Publication
(10) Pub. No.: US 2015/0117601 A1
(43) Pub. Date: Apr. 30, 2015

(54) X-RAY SOURCE WITH MODULE AND DETECTOR FOR OPTICAL RADIATION

(71) Applicants: Fraunhofer-Gesellschaft zur Förderung der angewandten Forschung e.V., München (DE); Charite - Universitätsmedizin Berlin, Berlin (DE)

(72) Inventors: Erwin Keeve, Potsdam (DE); Fabian Stopp, Neuenhagen (DE); Marc Käseberg, Biesenthal (DE); Sabastian Engel, Munster (DE); Eckart Uhlmann, Kiebitzreihe (DE)

(21) Appl. No.: 14/396,935

(22) PCT Filed: Apr. 23, 2013
(86) PCT No.: PCT/EP2013/058395

§ 371 Date: Oct. 24, 2014

(30) Foreign Application Priority Data
Apr. 25, 2012 (DE) 10 2012 008 812.4

(51) Int. Cl. A61B 6/08; A61B 6/00; A61B 6/02

(52) U.S. Cl. CPC A61B 6/08; A61B 6/022; A61B 6/5241
USPC 378/41

(57) ABSTRACT

The present invention relates to an X-ray source for irradiating an object to be imaged with X-ray radiation, wherein the X-ray source is moveable into a plurality of defined recording positions relative to the object to be imaged. A module is arranged directly on the X-ray source, said module being designed to project a marking onto the object to be imaged and/or to check a position of the object to be imaged and/or of the marking, wherein the module for checking the position of the object to be imaged and/or of the marking comprises a detector for optical radiation. In addition, the present invention relates to an X-ray device comprising the X-ray source and a method for using the X-ray device.

The present invention relates to an X-ray source with a module which is arranged directly on the X-ray source and with a detector for optical radiation. The present invention moreover relates to an X-ray device which comprises the mentioned X-ray source, and to a method with which the X-ray device is used.

X-ray devices serve for the destruction-free examination of objects to be examined. X-ray devices in the field of medicine are particularly applied for examining patients. X-ray radiation which is emitted by an X-ray source of the X-ray apparatus hereby partially penetrates the object to be examined, wherein absorption of the X-ray radiation occurs to a different extent depending on the through-radiated material. A share of the X-ray radiation which passes through the object is detected by a detector as a projection picture of the transilluminated object. Spatial information is represented in a superimposed manner in the projection picture, whereas a three-dimensional representation for example of interior of the body permits an exact reposition of bone breakages on joints or an exact positioning of implants relative to critical anatomical structures.

Several two-dimensional projection recordings of an object are taken from different spatial directions, and a scanned volume is subsequently reconstructed by way of an algorithm, for producing three-dimensional image data, by way of the X-ray device. With a medical application, this is effected before a surgical operation. A surgeon is mostly assisted by a navigation system during the operation. A planning as well as implementation of the operation is effected by way of preoperatively recorded picture data and the three-dimensional reconstruction, but it is necessary to plan and navigate by way of current picture data during the operation, on account of the anatomical structures of the patient which have changed during an operation and which are given due to a changed patient position or the due to the surgical operation which has already been partly carried out.

One solution which is known from the state of the art therefore envisages using a 3D X-ray system, such as a computer tomograph or a 3D C-arm, as well as a separate navigation system, in an operating theatre. Since conventional imaging systems encompass the patient, as a rule they cannot remain on the patient during the operation, which is why the navigation system and the imaging system are constructed as two separate systems in the operating theatre. The patient and the X-ray system are provided with localisers and are spatially localised via a camera of the navigation system, in order to carry out a navigated operation. If for example a 3D C-arm is used in combination with the navigation system, then the patient and the C-arm must be simultaneously localised by the camera of the navigation system. The C-arm is moved to the patient for a 3D scan, and is moved away from the patient again after the 3D scan. The manual alignment of the camera thereby is very cumbersome and a finished alignment must be corrected as the case may be, so that the localiser attached on the patient and the localiser of the X-ray system are simultaneously located in an operating region of the camera.

It is therefore the object of the present invention, to suggest an X-ray source and an X-ray device, which avoid the mentioned disadvantages, with which therefore an alignment of a camera of a navigation system is simpler to set and check. It is further the object of the invention to develop a method, with which the X-ray device is used.

According to the invention, this object is achieved by an X-ray source according to claim 1 as well as by an X-ray device according to claim 9 and by a method according to claim 11. Advantageous designs and further developments are specified in the dependent claims.

An X-ray source according to the invention which is designed, in order to irradiate an object to be imaged with X-ray radiation, is movable into several defined recording positions relative to the object to be imaged. A module is arranged directly on the X-ray source and is configured to project a marking onto the object to be imaged and/or to check a position of the object to be imaged and/or a position of the marking projected onto the object to be imaged. The module comprises a detector for optical radiation, for checking the position of the object to be imaged and/or of the marking.

An operating region of the module likewise includes the object to be imaged on account of the arrangement of the module directly on the X-ray source, inasmuch as the X-ray source is directed onto the object to be imaged. The module as a constituent (integral part) of a navigation system is thus combined with the X-ray source as a constituent of the imaging system. Thus the navigation system does not need to be constructed separately and a user of the X-ray source has free access to the object to be imaged, and the navigation system is constructed in a smaller and thus space-saving manner compared to known solutions. A cumbersome adjustment of the module is thus done away with. The module is also automatically co-moved by way of a movement of the X-ray source into one of the relative positions, so that in this case too, no renewed alignment needs to be effected. A possibility of feedback by way of checking the position of the object to be imaged or of the marking is rendered possible by way of the detector, by which means a readjustment of the relative position of the X-ray source becomes possible. The term “X-ray radiation” in the scope of this document is to be understood as an electromagnetic radiation with a wavelength of between 0.01 nm and 10 nm, whereas the term “optical radiation” in the scope of this document is to be understood as an electromagnetic radiation in the region visible to humans, between 400 nm and 700 nm, as well as in the regions of ultraviolet and infrared radiation which are adjacent to this.

In a further development, the detector for optical a radiation, although being arranged as a constituent of the module directly on the X-ray source, however can also be moved independently of the X-ray source. A larger angular region as a working region can be detected by the detector by way of this. A light source which is arranged in the module and is used for marking can likewise be moved independently of the X-ray source.

One can envisage the detector comprising a stereo camera which is configured to detect at least one tracker as a localiser, which is attached on the object to be imaged and/or on an instrument. Spatial information on the position of the X-ray source to the object to be imaged can also be obtained by the stereo camera. Moreover, the instruments which are applied on the object to be imaged and their positions can also be detected and processed.

The detector in a further embodiment can also comprise a surface scanner which is configured to scan a surface of the object to be imaged, said surface facing the X-ray source. Apart from the reconstruction of the information obtained from the projection pictures as X-ray data, additional information with regard to the surface of the object to be imaged is obtained by way of this, and this additional information assists a planning of a treatment of the object.

The module typically emits radiation in the visible wavelength region for projecting the marking, so that the user of the X-ray source can also perceive the radiation in a simple manner. Moreover, the detector can also thus detect the radiation. The module alternatively or additionally can also emit radiation in the infrared or in the ultraviolet region.

The module in one embodiment comprises a laser, since thus a coherent light source is provided with this, with which light source a point-accurate marking can be effected due to the typically small beam diameter. Moreover, a multitude of wavelengths can be used for marking, depending on the applied laser type. A laser radiation emitted by the laser in turn generally lies in the visible region, but can also lie in the infrared or ultraviolet region.

The marking which the module projects onto the object to be imaged, can be a laser-based target guidance line, by way of which the user is led to a defined point.

One can moreover envisage the module comprising a laser distance sensor. A distance between the X-ray source or the module and the surface of the object to be imaged can be determined by way of this and the planning and/or the projected marking can be adapted accordingly and specified in a more accurate manner.

The laser, the laser distance sensor, the stereo camera as well as the surface scanner are typical removably attachable onto the X-ray source and can be used individually or all together simultaneously. Moreover, also any combination of the mentioned components can be attached on the X-ray source and simultaneously used. A high variability of the X-ray source and of the module can be achieved by way of this, and these can be suitably retrofitted depending on the required application.

The X-ray source can be arranged on a holding device. The holding device preferably comprises an arm which can be moved in at least three, preferably in at least four, particularly preferably in at least five, typically however in six degrees of freedom. The degrees of freedom hereby generally have maximally three translatory and/or maximally three rotatory degrees of freedom. The X-ray source on the one hand is securely held by way of this, and on the other hand however can still be moved into infinite positions relative to the object to be imaged.

The X-ray source is typically fastened on the end of the arm, in order to ensure a maximal freedom of movement. The arm can be a robot arm or comprise this, wherein the robot arm is preferably movable in an automated manner via suitable joints, so that an activation of the arm movement is possible without the intervention of the user of the X-ray source. Inasmuch as the arm can be manually operated, the arm can comprise a force-moment sensor for assisting the positioning.

Preferably, the marking indicates an entry point as well as an entry angle for a treatment of the object, for example by way of one of the instruments. The module for this projects the marking at the entry angle onto the surface of the object to be imaged, i.e. the entry angle is defined as the angle between the surface of the object to be imaged and the marking which departing as a beam from the module is incident onto this surface, and can assume infinite values between 0° and 90°, preferably between 10° and 80°, particularly preferably between 30° and 60°. The instrument is applied on the projected entry point and is positioned until the projected marking lies on an end piece of the instrument, for aligning onto the object to be imaged. In this position, the instrument is arranged on the object to be imaged at the desired entry angle.

Typically, the object to be imaged is a patient and the X-ray source is used in an operating theatre by the surgeon in a preoperative and/or intraoperative manner. The instrument in this case is preferably a surgical instrument, which is particularly preferably provided with an instrument tracker. The planning in this case includes the entry point of the surgical instrument on the surface of the skin of a patient and of a target structure in the inside of the patient.

An X-ray device according to the invention preferably comprises a X-ray source with the previously described characteristics, an X-ray detector for detecting the X-ray radiation as a projection picture, an evaluation unit for evaluating the projection pictures which have been taken by the X-ray detector, wherein the evaluation includes a computation of a reconstruction of a target region recorded on the projection pictures, an output unit for outputting (issuing) the projection picture or projection pictures and/or the reconstructions, as well as a control unit for activating the X-ray source, the X-ray detector, the evaluation unit and the output unit. By way of this, projection pictures as X-ray recordings can be made, processed further and evaluated, and the checking of the position and/or projecting of the marking be carried out. The usually separately provided navigation system is this combined with the system for the three-dimensional imaging. The X-ray device according to the invention comprises at least the X-ray source with the previously described characteristics and the X-ray detector for detecting the X-ray radiation as a projection picture.

Typically, the X-ray source and the X-ray detector are spatially separated from one another and are preferably not directly mechanically connected to one another. A free access to the object to be imaged for the user, typically the surgeon, exists during or between recordings on account of this. Moreover, the X-ray source and the X-ray detector are arranged relative to one another in a manner such that the X-ray radiation which is emitted by the X-ray source is incident onto the X-ray detector in an orthogonal or non-orthogonal manner.

The X-ray source is preferably movable, typically movable in an automated manner, independently of the X-ray detector. The X-ray detector alternatively or additionally can also be movable, preferably movable in an automated manner, independently of the X-ray source.

The output (issuing) of the projection pictures and/or of the reconstructions can include a representation on a screen. Alternatively or additionally, the output can also be effected on a printer. The user thus receives information on the object to be imaged, in a simple and unambiguous perceivable manner. The reconstructions are hereby preferably three-dimensional.

The control unit can be designed to control the movement of the X-ray source and/or of the module, as well as the position of the marking and the recording of the projection pictures, in an automated manner. This is preferably effected via a control computer, which comprises software for carrying out the mentioned tasks as well as planning software. A treatment of the object to be imaged can be planned with the help of planning software, and the user accordingly instructed with regard to the treatment. Hints with regard to the treatment for this are particularly preferably likewise represented on the output unit. Typically, the control unit and the evaluation unit are combined into one apparatus, in order to save space.

Inasmuch as the X-ray source comprises the already described stereo camera, the X-ray device comprises at least one tracker which is to be attached on the object to be imaged and/or on one of the instruments and which is detected by the stereo camera. The position of the object to be imaged or the position of the respective instrument can be simply detected by way of this and used for planning. The stereo camera can additionally or alternatively also track the light source projected by the module.

Typically, an evaluation of the positions of the X-ray source and of the X-ray detector as well as of the object to be imaged is effected in a base coordinate system, whose origin is preferably defined by the object to be imaged. The positions of the individual components are clearly defined, and simple to transform, on account of the base coordinate system.

A method, by way of which the object to be imaged is checked and/or marked by way of the X-ray device with the described characteristics, comprises several steps. In one step, at least two projection pictures of the object to be imaged are taken from different directions. For recording, the object to be imaged at least partly is through-beamed by X-ray radiation, which depending on the composition of materials contained in the object to be imaged, is absorbed to a different extent. Shares of the X-ray which are not completely absorbed run through the object to be imaged and with their respective intensity are detected by the X-ray detector.

In a further step, a three-dimensional reconstruction of the recorded object to be imaged is computed from the recordings. Finally, a target region defined in the three-dimensional reconstruction or the object to be imaged, by way of the module arranged on the X-ray source is marked and/or a position of the object to be imaged and/or of the marking checked. The object to be imaged can be reliably detected and reproduced as well as marked and checked by way of this, so that the user receives detailed information with regard to further procedure.

Typically, a first marking of the object to be imaged and a first detection of the marking are effected already before the recording of the at least two projection pictures, so that the X-ray source can be aligned by way of the marking.

The reconstruction of the object to be imaged is preferably effected by way of an iterative algorithm, so that the reconstruction of the recorded object is rendered possible with projection pictures from infinite directions.

Typically, in each case the entry point and the entry angle on the object to be imaged is displayed (indicated) by way of the reconstructions, so that the user is instructed on using the instruments.

The checking of the position of the target region is preferably effected already during the taking of the projection pictures, so that position changes can already be ascertained at an early stage and suitable corrections carried out.

Preferably, an adaptation of an alignment of the stereo camera for tracking one of the trackers is preferably effected in a continuous manner, inasmuch as the stereo camera is used. The trackers which are arranged on the object to be imaged or one of the instruments are therefore always in the operating region of the stereo camera and are detected by this.

Particularly preferably, in one method step, after the checking of the position of the object to be imaged, the alignment of the X-ray source relative to the object to be imaged is adapted in a manner depending on the ascertained position of the object to be imaged. This is typically effected in an automated manner.

Moreover, one can also envisage the recording of the projection pictures and the projection of the marking being effected in real-time, so that the user of the X-ray device immediately obtains a feedback with regard to the further procedure.

If trackers are used, then a relative position of the X-ray source to one of the trackers can be stored and moved to afresh at a later point in time. Thus a recording from a same position is also possible with an intermediate movement of the object to be imaged.

Only the target region as a part of the object to be imaged can be also imaged, instead of completely recording the object to be imaged. An unnecessary radiation exposure of the remaining object is avoided by way of this. The target region, which is also indicated as the target structure, is typically defined in one of the three-dimensional reconstructions.

If with regard to the object to be imaged, it is the case of a patient, in a further method step which is generally carried out before the beginning of the X-ray recordings, a patient registration can be effected, with which the data of the patient is detected and is linked to the projection pictures which are subsequently recorded. The data thereby includes position information of the three-dimensional reconstruction as well as position information which is determined from the video camera or of the surface scanner. The patient registration describes a transformation between the 3-D picture data of the patient and a current position of the patient in space and permits the saving of time when planning and carrying out the treatment of the object.

A calibration with which position information, thus positions of the X-ray source and of the X-ray detector, are defined in the base coordinate system is preferably effected as a first step of the method.

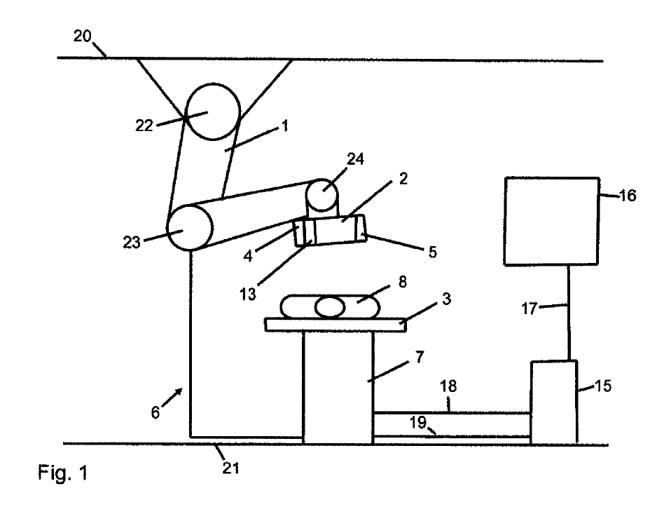

Embodiment examples of the invention are represented in the drawings and are hereinafter described by way of FIGS. 1 to 3.

There are shown in:

FIG. 1 a lateral view of an X-ray device with an X-ray source and with an X-ray detector,

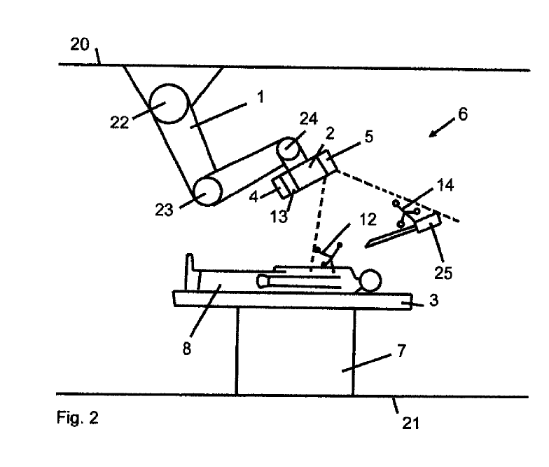

FIG. 2 the X-ray device shown in FIG. 1, with an instrument, in a view which is rotated by 90° with respect to the view of FIG. 1 and

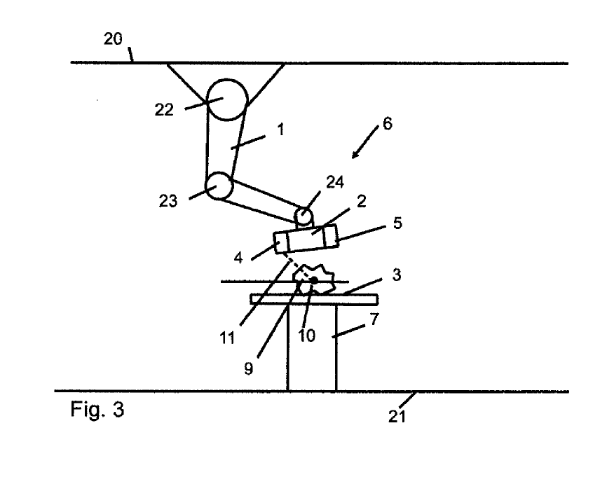

FIG. 3 a further embodiment example of the X-ray device which is represented in FIG. 1.

FIG. 1 in a lateral view shows an X-ray device 6 in an operating theatre. The X-ray device 6 is designed for the generation of volume data of a patient 8 as an object to be imaged, during a surgical operation and for an immediate provision of navigation information on the basis of current patient data. Medical fields of application for this are for example accident surgery, oral surgery, orthodontic and facial surgery, orthopaedics, neurology, urology and cardiology.

The X-ray source 6 comprises an X-ray source 2 which is fastened at one end of a robot arm 1, an X-ray detector 3 as well as a computer 15 which serves as an output unit as well as a control unit. Moreover, the X-ray device 6 comprises a screen 16 as an output unit. The computer 15 is connected to the screen 16 via a cable 17. The computer 15 moreover is connected to the X-ray source 2 and to the X-ray detector 3, in each case via a cable 18, 19. The computer 15 is also connected to the robot arm 1 via the cable 18 and can move this robot arm in an automated manner. Instead of the connection via cables 17, 18, 19, the computer 15 can also be connected to the screen 16, the X-ray detector 3 and to the X-ray source 2 in a wireless manner. The computer 15 comprises picture recording software, display software, 3D reconstruction software, planning software and control software.

The robot arm 1 is fastened on a ceiling 20 of the operating theatre, but in further embodiments can of course also be attached on the floor 21. The robot arm 1 comprises three joints 22, 23, 24, by way of which the robot arm 1 and thus the X-ray source 2 fastened on the end of the robot arm can be moved in total in six degrees of freedom, three translatory degrees of freedom and three rotatory degrees of freedom. Moreover, the robot arm 1 comprises a force-moment sensor, in order to move and align the X-ray source 2 also via a manual force effect or manual force guidance. The X-ray source 2 can be moved by way of the robot arm 1 into several relative positions to the X-ray detector 3. The relative positions can lie above, below but also laterally of the X-ray detector 3. Instead of on the robot arm 1, the X-ray source 2 can also be fastened on a stand arm which for example is movable on a vehicle.

A module which comprises a stereo camera, 5, a laser light module 4 having a laser distance sensor, and a surface scanner 13 is fastened directly on the X-ray source 2. The module is removably attachable onto the X-ray source 2, wherein the components of the module, thus the stereo camera 5, the laser light module 4 and the surface scanner 13 can also be fastened individually or in infinite combinations, on the X-ray source 2. The module bears on the X-ray source 2 in a flush manner, so that no corners or edges are formed. In further embodiments, a conventional camera as a detector for optical radiation can also be used instead of a stereo camera 5.

The X-ray source 2 is designed to emit X-ray beams, which are detected by the X-ray detector 3. The X-ray detector 3 lies on a patient table 7. The patient table 7 is fixedly connected to the base 21 of the operating theatre. The patient 8 as an object to be imaged lies on the X-ray detector 3, so that the X-ray source 2 by way of moving the robot arm 1 is also moved into several relative positions to the patient 8. The X-ray detector 3 in further embodiments can also be removably attachable onto the patient table 7 and moreover the X-ray detector 3 can also be arranged below a patient rest, wherein the patient 8 lies on the patient rest.

Likewise, the X-ray detector 3 can be arranged on a positioning device which permits a movement of the X-ray detector with respect to the patient 8. The positioning device can preferably comprise at least one side element which can be angled and/or an articulated arm, in order to permit a displacement of the detector into several recording positions which are different, or at least do not all lie on a straight line, wherein at the same time an unambiguous assignment of the X-ray images to positions of the detector is possible by way of the positioning device. The German patent application 10 2012 005 899.3 is referred to for this, with regard to the arrangement of the X-ray detector as well as the design of the positioning device, and this application is thus incorporated as a constituent into the present application. Likewise, all embodiment examples of the mentioned patent application which has been referred to, and which are supplemented by the module 4, 5, 13 designed according to the invention, are to be considered as being disclosed.

The X-ray beams which are emitted by the X-ray source 2 run through the patient 8 and are absorbed in the body of the patient 8 to a different extent. A share of the X-ray beams which has not been completely absorbed finally hits the X-ray detector 3. The X-ray detector 3 is a semiconductor-based flat detector which transmits a picture obtained by the incident X-ray radiation, to the computer 15 via the cable 18.

The computer 15 processes the picture, typically a projection recording, and outputs the processed picture in real-time on the screen 16. Inasmuch as several projection recordings of the patient 8 or also of only a target region of the patient such as individual organs are present, the computer 15 carries out a three-dimensional reconstruction of the recorded region preferably with an iterative algorithm and also represents this reconstruction in real-time on the screen 16. A target region is determined by way of the reconstruction and is marked by the module.

Positional information of the X-ray source 2 and of the X-ray detector 3 in a base coordinate system is known by way of a calibration. The base coordinate system is an orthogonal coordinate system whose origin lies in the centre of gravity of the patient 8. Alternatively, the origin can also lie at a corner of the X-ray detector 3 or in the base of the robot arm. A calibration was carried out before the taking the projection recordings or pictures, with which calibration positions of the X-ray source 2 and of the X-ray detector 3 in the base coordinate system were determined. The X-ray source 2 is moved into different relative positions to the patient by way of moving the robot arm 1, so that X-ray recordings at different angles are possible. The three-dimensional reconstruction can then be effected on the basis of positional information of the X-ray source 2 and of the X-ray detector 3, said information being known in the base coordinate system. The position of the target region as a reconstructed volume is finally also known in the base coordinate system.

The information for the navigation of a surgeon on a patient 8 which is necessary for the operation is therefore obtained without a moving of the patient table 7, thus without a movement of the patient 8 after taking the projection recordings.

The X-ray device 6 of FIG. 1 is represented in FIG. 2 in a lateral view which has been rotated by 90° with respect to the view of FIG. 1. Recurring features in this as well as in the following figures are provided with the same reference numerals.

The stereo camera 5 is designed as a detector for optical radiation (for example in a wavelength region between 400 nm and 1 mm) and measures the position of trackers 12, 14 in real-time. The position of the stereo camera 5 in the base coordinate system is known by way of the calibration, but however the stereo camera 5 on the X-ray source 2 can be moved independently of the X-ray source 2. The patient 8 and any instruments which are used by the surgeon during the operation are provided with the trackers 12, 14 as localisers. The trackers 12, 14 are formed from a structure which reflects optical radiation. The stereo camera 5 localises one of the trackers 12 which is attached as a patient tracker on the patient 8, and this localisation takes place before, during and after the taking of the projection recordings. One checks as to whether the position of the patient 8 has changed by way of this. The further tracker 14 is attached as an instrument tracker on the instrument used by the surgeon, and is likewise detected by the stereo camera 5.

An automatic patient registration is carried out by way of the known position information of the three-dimensional picture data and the position data of the tracker 12 which is attached on the patient 8, said position data being determined by the stereo camera 5. For this, the computer 15 transforms the picture data of the patient 8 and the position of the patient 8 in the base coordinate system. The navigation software in the computer 15 can automatically display a position of the instruments 25 of the surgeon and which are provided with the trackers 14, in the recorded three-dimensional picture data of the patient due to the patient registration and visually assist the surgeon with the operation by way of a display on the screen 16.

The stereo camera 5 and the tracked instrument 25 which in the embodiment example shown in FIG. 2 is a pointer instrument, are used for ascertaining the target region for a (as the case may be further) three-dimensional picture data recording. A determining of the target region by way of the tracked instrument 25 can be effected with or without a present patient registration. The surgeon scans an anatomic structure in situ of the patient 8 with the tracked pointer instrument 25. A position of a dial of the instrument 25 is measured with the stereo camera 5. The picture data recording is subsequently carried out in a manner such that the scanned target region is located in the centre of the scanned volume and therefore also in the centre of the generated reconstructions as 3D X-ray picture data. Moreover, a relative position to the scanned anatomical structure can also be specified without a cumbersome alignment of the imaging system.

The stereo camera 5 is moved in an automated manner for the optimal alignment of this. A working region of the stereo camera 5 is optimally utilised or a working region of the surgeon is optically imaged by way of this. It is also possible to align the stereo camera 5 onto a certain one of the trackers 12, 14 and to track this, as the case may be also during its movement and hereby to continuously adapt the alignment of the stereo camera 5. The surgeon during the operation has a free access to the patient 8 and thus can also freely move the instrument and the instrument tracker 14 which is attached thereto, due to the spatial separation of the X-ray source 2 and of the X-ray detector 3. The X-ray radiation which is emitted by the X-ray source 2 in a cone-like manner is incident on the X-ray detector 3 at any angle, wherein a cone central beam is typically orthogonally incident onto the X-ray detector 3.

After the stereo camera 5 has once been aligned to the patient 8 during the taking of a two-dimensional projection recording, this relative position of the X-ray source 2 with regard to the patient 8 and the X-ray detector 3 in the base coordinate system can be stored. If a further recording is to be made from this position at a later point in time of an operation, the robot arm 1 automatically brings the X-ray source 2 into the same position with respect to the tracker 14 attached on the patient 8. The robot arm 1 hereby is controlled by the computer, by way of the position being stored. The original recording can be repeated by way of this, without a manual renewed alignment of the X-ray device 6 having to be carried out, despite a possible movement of the patient 8 effected in the meantime.

With a covering of the tracker 12, 14 during the surgical operation, for example by way of further necessary instruments, the alignment of the stereo camera 5 can be optimised via the robot arm 1 which is controlled by the computer 15, in order to create better viewing conditions between the stereo camera 5 and the trackers 12, 14. Location information of the trackers 12, 14 before the covering for this is set into relation with surface data during the covering, said data being obtained by the surface scanner 13. The surface scanner 13 for this can be moved in an automated manner independently of the X-ray source 2. The movement of the surface scanner 13 just as the picture recording by way of the X-ray source 2 and the movement of the X-ray source 2 can be controlled by the computer 15. The laser light module 4 is also activated, and the laser beam 11 faded in or out, by way of the control unit. A suitable position and alignment of the stereo camera 5 is determined by way of the position information before the covering and the depth information on covering, from which position and alignment it can directly record the trackers 12, 14. The stereo camera 5 can be adjusted with regard to its height for this.

In the case that the module comprises no stereo camera 5, the patient registration can also be effected only with the help of the surface scanner 13 and be updated during the operation. For example, for operations with oral, orthodontic and facial surgery, a facial surface of the patient 8 can be scanned and can be brought into relation to the skin surface extracted from the recorded 3D picture data. The surface scanner 13 is generally configured to scan a surface of the patient 8 which faces it.

FIG. 3 in a lateral view corresponding to FIG. 1 shows a further embodiment of the X-ray device 6, with which the module which is arranged on the X-ray source 2 only comprises the stereo camera 5 and the laser light module 4. The laser light module 4 comprises a laser as a light source, which emits a laser beam 11 in the visible wavelength region. A laser point or laser cross hairs can be faded in as a marking, by way of the laser beam 11. The laser beam 11 however can also serve as a target guidance line, in order to lead the surgeon to a certain region to be treated. Alternatively or additionally, the laser-light module 4 can also emit a laser beam in the infrared or ultraviolet wavelength region. The marking can indicate the target region, wherein the stereo camera 5 checks the position of the patient 8 and of the marking. The laser beam 11 is blended in as a first marking on the patient 8 already before taking the first projection recordings. The stereo camera 5 also checks the position of the patient 8 during the taking of the projection recordings or pictures. The alignment of the X-ray source 2 relative to the patient 8 is automatically adapted in dependence on the position of the patient 8 which is detected by the stereo camera 5.

The laser light module 4 just as the stereo camera 5 is movable independently of the X-ray source 2, so that the laser beam 11 is emitted at different angles to the X-ray source 2. The position of the laser light module 4 and the laser beam 5 are known in the base coordinate system. An entry point and an entry angle 9 to a target region 10 or target region of the patient 8 can be planned in the 3D picture data by way of navigation software stored in the computer 15. The laser beam 11 is incident onto the entry point on the surface of the patient 8 at the entry angle 9 which in the present embodiment example is 45°. The target region 10 is located in the inside of the patient 8 and in the embodiment example represented in FIG. 3 is the lung. A biopsy needle is applied onto the entry point and is rotated until the laser beam 11 is incident on a grip of the biopsy needle, in order to get to the target region 10. The biopsy needle is thus at the correct position and has the correct alignment for the biopsy.

Directly subsequently to the 3D picture recording, which contains the target region, and a subsequent planning, the computer 15 via the robot arm 1 aligns the laser light module 4 in a manner such that the laser beam 11 indicates or displays the entry point and the entry angle 9. The patient 8 hereby is not moved between the 3D picture recording and the target guide display of the laser light module 4.

Inasmuch as the X-ray device 6, as is represented in FIG. 3, is additionally provided with the stereo camera 5, the patient 8 can be moved between the 3D picture data recording, the planning and the target guidance display. The computer 15 controls the robot arm 1 in a manner such that the movement of the patient 8 is automatically compensated by way of a suitable position change of the laser light module 4 with respect to the tracker 12.

The laser light module 4 moreover comprises the laser distance sensor, which is used for the distance measurement between the X-ray source 2 and the patient 8. This simplifies the navigation and the alignment. The spatial coordinates of the point, onto which the laser beam 11 is focussed, can be determined in the base coordinate system by way of the position and orientation of the robot arm 1 in combination with the laser distance sensor.

The laser light module 4 is used for aligning the X-ray source 2 or the stereo camera 5 on the robot arm 1. The laser cross hairs as a marking, for this are firstly focussed onto the target region. As the case may be, several alignments from different directions are necessary for aligning the 3D picture data recording. Additionally, a relative position to the structure focussed with the laser distance sensor can be specified, for example a depth.

The laser cross hairs or the laser point can be focused onto a further structure after the 3D picture recording. This structure or this point is displayed in the three-dimensional reconstructions, in order render the identification or orientation in situ simpler for the surgeon.

Inasmuch as features have only been disclosed in individual ones of the embodiment examples, no limitation to the use with this embodiment is to be seen therein. In contrast, these features can also be combined with other embodiment examples and commonly claimed.

1. An X-ray source for irradiating an object to be imaged, with X-ray radiation,
wherein the X-ray source is movable into several defined recording positions relative to the object to be imaged, and
wherein a module is arranged directly on the X-ray source and is configured to project a marking onto the object to be imaged and/or to check a position of the object to be imaged and/or of the marking, and
wherein the module comprises a detector for optical radiation, for checking the position of the object to be imaged and/or of the marking.

2. The X-ray source according to claim 1, wherein the detector comprises a stereo camera for the detection of at least one tracker which is attached on the object to be imaged and/or on an instrument, and/or of the marking,

3. The X-ray source according to claim 1, wherein the detector is movable independently of the X-ray source, preferably in an automated manner.

4. The X-ray source according to claim 1, wherein the detector comprises a surface scanner for scanning a surface of the object to be imaged, said surface facing the X-ray source.

5. The X-ray source according to claim 1, wherein the module emits radiation in the visible wavelength region, for projecting the marking.

6. The X-ray source according to claim 1, wherein the module comprises a laser.

7. The X-ray source according to claim 6, wherein the laser is movable independently of the X-ray source, preferably in an automated manner.

8. The X-ray source according to claim 1, wherein the module is a laser distance sensor.

9. The X-ray source according to claim 1, wherein the X-ray source is arranged on a holding device which comprises an arm which can be moved in an automated manner in at least four degrees of freedom.

10. The X-ray source according to claim 9, wherein the degrees of freedom include at least one translatory degree of freedom.

11. The X-ray source according to claim 1, wherein the marking indicates an entry point and an entry angle, at which the marking hits the object to be imaged, for a treatment of the object to be imaged.

12. The X-ray device comprising an X-ray source according to claim 1, and an X-ray detector for detecting the X-ray radiation as a projection picture.

13. The X-ray device according to claim 12, further comprising an evaluation unit for evaluating the projection pictures taken by the X-ray detector, by way of computing a reconstruction of a target region which is recorded on the projection pictures, by an output unit for outputting the projection pictures and/or the reconstructions, and by a control unit for the control of the X-ray source, the X-ray detector, the evaluation unit and the output unit.

14. The X-ray device according to claim 12, wherein the X-ray device, in the case that the X-ray source comprises a stereo camera, comprises at least one tracker which is to be attached on the object to be imaged and which is detected by the stereo camera.

15. The X-ray device according to claim 12, wherein the X-ray source is movable independently of the X-ray detector, preferably in an automated manner, and/or that the X-ray detector is movable independently of the X-ray source, preferably in an automated manner.

16. The X-ray device according to claim 12, wherein the X-ray source and the X-ray detector are spatially separated from one another and are not directly mechanically connected to one another.

17. A method for checking and/or marking an object to be imaged, by way of an X-ray device according to claim 12, the method comprising:
recording at least two projection pictures of the object to be imaged;
computing a three-dimensional reconstruction of the recorded object to be imaged; and
marking a target region defined in the three-dimensional reconstruction by way of the module arranged on the X-ray source and/or checking a position of the object to be imaged and/or of the marking.

18. The method according to claim 17, wherein a marking of the object to be imaged and a detection of the marking are effected before the taking of the at least two projection pictures.

19. The method according to claim 17, wherein the marking indicates the entry point and the entry angle on the object to be imaged, said entry point and entry angle being ascertained in each case by way of the reconstruction.

20. The method according to claim 17, wherein a checking of the position of the object to be imaged is effected already during the taking of the projection pictures.

21. The method according to claim 17, wherein inasmuch as the stereo camera is used, an alignment of the stereo camera is constantly adapted for tracking one of the trackers.
